(19) United States
(12) Patent Application Publication
(10) Pub. No.: US 2010/0185147 A1
(43) Pub. Date: Jul. 22, 2010

(54) DISPOSABLE SAFETY SYRINGE STRUCTURE

(76) Inventor: Cho-Ying Chen, Shengang Shiang (TW)

Correspondence Address: Wang Law Firm, Inc., 4989 Peachtree Parkway,, Suite 200, Norcross, GA, 30092, US

(21) Appl. No.: 12/692,524

(22) Filed: Jan. 22, 2010

(30) Foreign Application Priority Data
Jan. 22, 2009 (TW) 098201291

(51) Int. Cl. A61M 5/50

(52) U.S. Cl. 604/110

(57) ABSTRACT

A disposable safety syringe structure provides a safe application of an injection syringe and comprises a barrel body having a positioning seat and a latch slot, and the needle seat includes an inverted hook plate, a protruding pillar, a latch block, a push rod, and a plunger having a circular flange, such that when a liquid medicine is injected, the latch block of the protruding pillar of the needle seat is combined with the latch slot of the positioning seat of the barrel body, and after the liquid medicine is injected, the plunger of the push rod is forced to be plugged into the needle seat integrally, and the latch block of the protruding pillar is separated from the latch slot of the positioning seat, and the needle seat together with the injection needle are hidden into the barrel body to prevent the injection needle from piercing users and others.

### BACKGROUND OF THE INVENTION

1. Field of the Invention

The present invention relates to a disposable safety syringe structure, and more particularly to an improved safety syringe structure having a needle seat of an injection needle installed in a barrel body, such that after a liquid medicine is injected, a plunger of a push rod can be plugged into the needle seat and coupled integrally with the needle seat, and the needle seat can be detached and slid to achieve the effect of hiding the needle seat and its injection needle into the syringe, so as to prevent users and others from being pierced and injured by the injection needle and provide a convenient use of a safety syringe.

2. Description of the Related Art

As conventional disposable safety syringe structures generally have the issues of a complicated stricture and a high manufacturing cost. Furthermore, after a liquid medicine is injected, and a plunger of a push rod connected to a needle seat of an injection needle are retracted, there is a relatively large resistance with internal walls of the barrel body, and thus the connected structure of the conventional plunger and needle seat may be detached by mistake. Furthermore, it is necessary to apply a large force such as pressing the injection needle against a desktop in order to push the push rod for the application, and users or others may be pierced or injured by the injection needle easily if the pushing of the push rod slips.

### SUMMARY OF THE INVENTION

In view of the shortcomings of the prior art with a complicated structure and an inconvenient use, the inventor of the present invention based on years of experience in the related industry to conduct extensive researches and experiments, and finally developed a disposable safety syringe structure in accordance with the present invention to simplify the structure and lower the cost.

Therefore, it is a primary objective of the present invention to overcome the aforementioned shortcomings and deficiencies of the prior art by providing a disposable safety syringe structure as shown in FIGS. 1 to 4, and the disposable safety syringe structure comprises a barrel body 10, a positioning seat 11 disposed in the barrel body 10 and provided for installing a needle seat 20, and a plunger 31 disposed at a distal end of a push rod and having a circular flange 33, such that after a liquid medicine is injected, and the push rod 30 is pushed to drive the plunger 31 to be plugged into the needle seat 20, the plunger 31 and the needle seat 20 will be combined integrally, and the needle seat 20 will be detached from the positioning seat of the barrel body 10, such that the needle seat 20 together with the injection needle 28 are retracted and hidden into the barrel body 10, so as to achieve the effects of providing a convenient and quick application, hiding the injection needle 28 into the barrel body 10 easily, and preventing users and others from being pierced and injured by the injection needle.

### BRIEF DESCRIPTION OF THE DRAWINGS

FIG. 1 is an exploded view of the present invention;

FIG. 2 is a cross-sectional view of the present invention;

FIG. 3 is a cross-sectional view showing a status of filling up a liquid medicine for injection in accordance with the present invention; and

FIG. 4 is a cross-sectional view showing a status of retracting an injection needle into a barrel body in accordance with the present invention.

### DETAILED DESCRIPTION OF THE PREFERRED EMBODIMENTS

The technical characteristics of the present invention will become apparent with the detailed description of the preferred embodiments and the illustration of the related drawings.

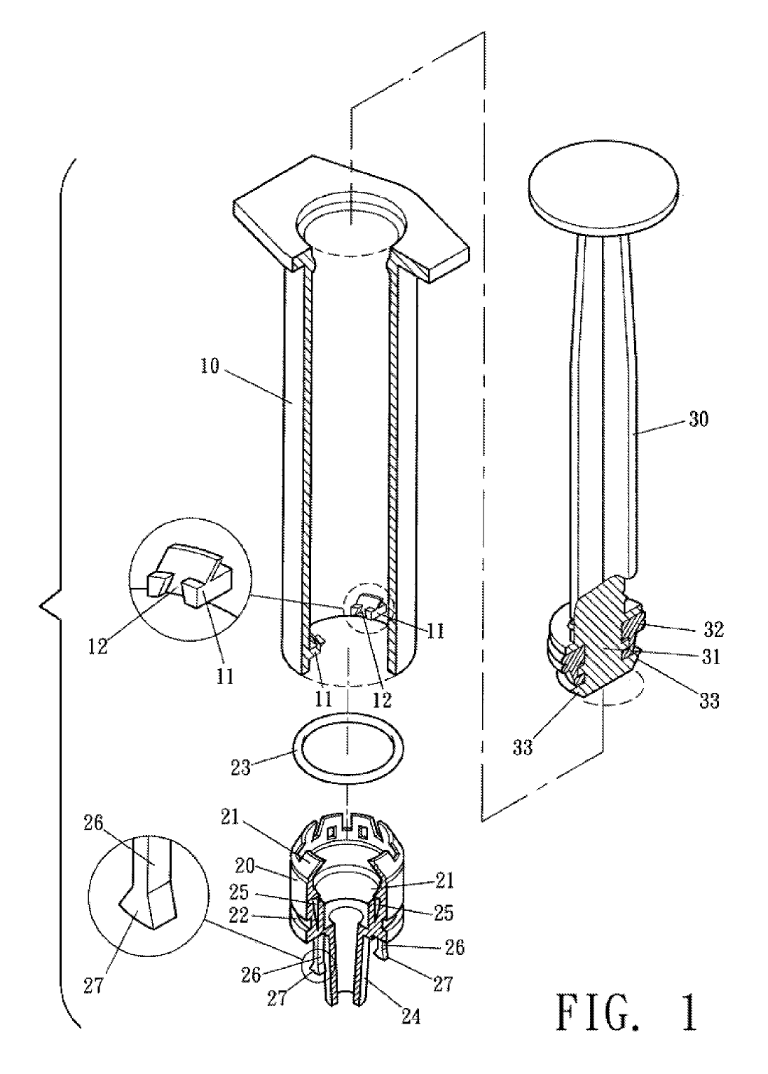

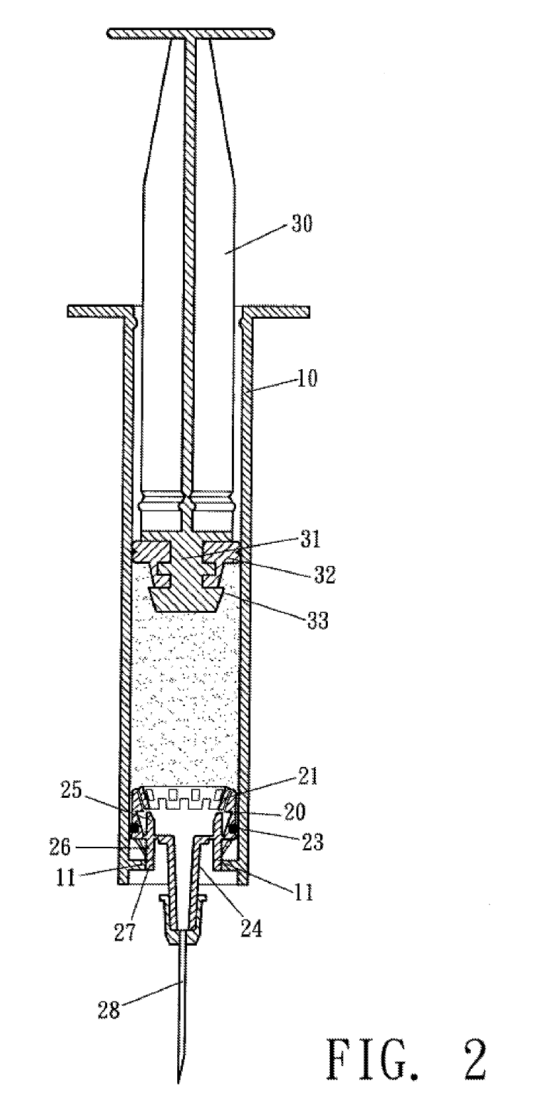

With reference to FIGS. 1 and 2, the disposable safety syringe structure comprises a barrel body 10, a needle seat 20, a push rod 30, a plunger 31, a positioning seat 11 disposed inside a distal end of the barrel body 10, a latch slot 12 formed and disposed on the positioning seat 11, a sheath section 24 disposed at a front end of the needle seat 20 for combining an injection needle 28, an elastic inverted hook plate 21 with an inwardly folded at a rear end, and a circular groove 22 disposed around the needle seat 2 for combining an anti-leak ring 23. In addition, a protruding pillar 26 is disposed at a front end of the needle seat 20, and a latch block 27 is protruded separately from ends of both opposites sides of the protruding pillar 26, and the needle seat 20 contains a guide pillar 25 integrated with the protruding pillar 26, such that when the needle seat 20 is installed into the barrel body 10, the latch block 27 of the protruding pillar 26 is latched with the latch slot 12 of the positioning seat 11 of the barrel body 10 precisely to achieve a fixing effect, and the plunger 31 of the push rod 30 includes a piston gasket 32 installed thereon and a circular flange 33 disposed at a front end of the push rod 30.

With the aforementioned structure, the present invention can achieve the following effects and functions:

1. In FIG. 2, a normal status of injecting a liquid medicine is shown, wherein the needle seat 20 is installed at the positioning seat 11 of the barrel body 10, and the elastic inverted hook plate 21 has be folded into a ready status, and the piston gasket 32 of the plunger 31 of the push rod 30 can be push to inject the liquid medicine.

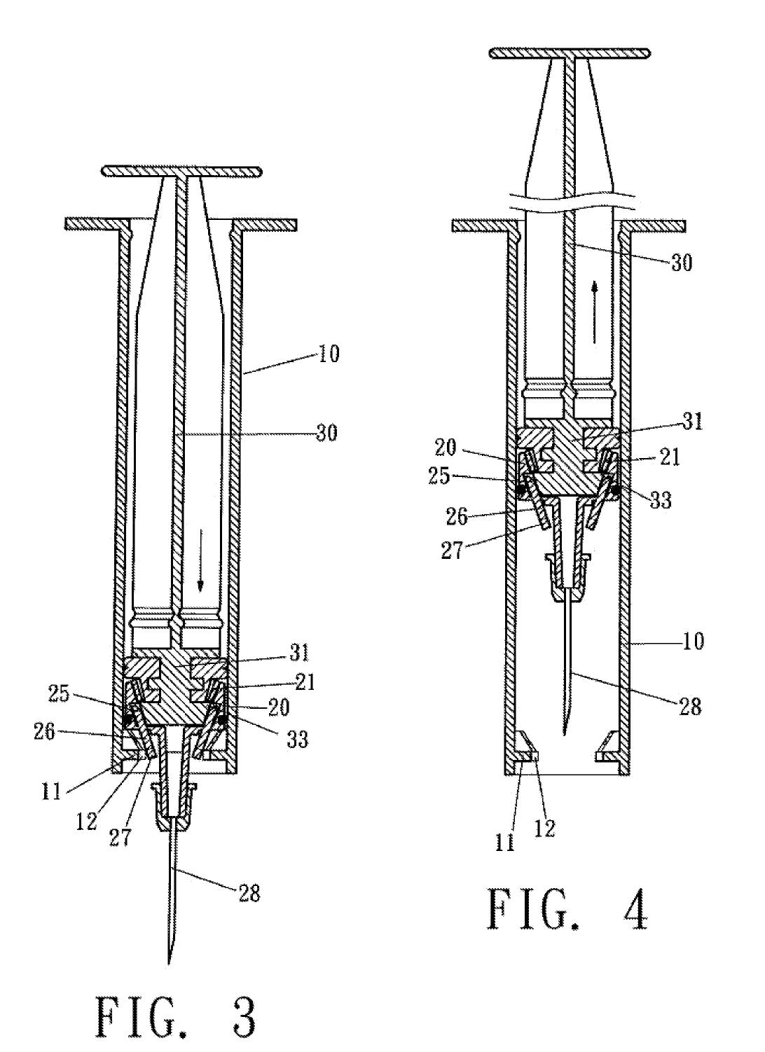

2. In FIG. 3, a status of completing the injection of the liquid medicine is shown. Now, a force is applied to push the push rod 30 to move forward, and the folded inverted hook plate 21 of the needle seat 20 is titled to an inclination and elastically expanded, such that the plunger 31 is forced and plugged into the needle seat 20. Since the circular flange 33 of the plunger 31 is also tilted into an inclination accordingly, the plunger 31 is pushed forward, such that the circular flange 33 will be in contact with an oblique surface of the guide pillar 25, and the guide pillar 25 is pushed outward. Due to a lever effect, the protruding pillar 26 of the needle seat 20 is forced to move slantingly. In other words, the latch block 27 of the protruding pillar 26 is separated from the latch slot 12 of the positioning seat 11 of the barrel body 10, and the circular flange 33 of the plunger 31 is hooked and latched with the folded inverted hook plate 21 of the needle seat 20 to combine the plunger 31 and the needle seat 20 integrally.

3. In FIG. 4, the latch block 27 of the protruding pillar 26 of the needle seat 20 is separated from the latch block 12 of the positioning seat 11 of the barrel body 10, so that when the plunger 31 of the push rod 30 is retracted, the needle seat 20 together with the injection needle 28 are retracted and hidden into he barrel body 10 to prevent the injection needle 28 from piercing and injuring users and others.

In summation of the description above, the present invention comes with a simple structure, such that when a user wants to store and hide the injection needle 28, the user simply needs to push the push rod to combine the plunger 31 with the needle seat 20 in a convenient and quick manner, and the connection of the plunger 31 and the needle seat 20 provides a full periphery latch of the circular flange 33 and the circular inverted hook plate 21 to resist a large pulling force and prevent any detachment by mistake, so as to assure that the retracted push rod 30 can link and retract the needle seat 20. The present invention improves over the prior art and complies with patent application requirements, and thus is duly filed for the patent application.

While the invention has been described by device of specific embodiments, numerous modifications and variations could be made thereto by those generally skilled in the art without departing from the scope and spirit of the invention set forth in the claims.

What is claimed is:

1. A disposable safety syringe structure, comprising a barrel body, a needle seat, a push rod, a plunger, a positioning seat disposed in a distal end of the barrel body, a latch slot formed on the positioning seat, a sheath section disposed at a front end of the needle seat for combining an injection needle, an elastic inverted hook plate disposed at a rear end of the needle seat and having a circular groove formed around the periphery for combining an anti-leak ring, a protruding pillar disposed at a front end of the needle seat, a latch block protruded separately from an end of both opposite sides of the protruding pillar, such that when the needle seat is installed into the barrel body, the latch block of the protruding pillar is latched precisely into the latch slot of the positioning seat of the barrel body to achieve a fixing effect, a piston gasket installed onto the plunger of the push rod, and a circular flange disposed at a front end of the plunger, such that after a liquid medicine is injected, the plunger is forced to be plugged into the needle seat, and the circular flange of the plunger is hooked and latched with the inverted hook plate of the needle seat to integrate the plunger with the needle seat, and the plunger being provided for separating the latch block of the protruding pillar of the needle seat from the latch slot of the positioning seat of the barrel body, so as to retract the plunger of the push rod plunger and drive the needle seat together with the injection needle to be retracted into the barrel body.
